(19) United States
(12) Patent Application Publication
(10) Pub. No.: US 2006/0241571 A1
(43) Pub. Date: Oct. 26, 2006

(54) Energy irradiation device and energy irradiation method

(75) Inventors: Norihiko Hareyama, Hino-shi (JP); Taisuke Sato, Hachioji-shi (JP); Tomoyuki Takashino, Hino-shi (JP)

Correspondence Address: OSTROLENK FABER GERB & SOFFEN, 1180 AVENUE OF THE AMERICAS, NEW YORK, NY, 100368403, US

(73) Assignees: Olympus Corporation; Olympus Medical Systems Corporation

(21) Appl. No.: 11/451,826

(22) Filed: Jun. 13, 2006

Related U.S. Application Data
(63) Continuation of application No. PCT/JP05/08046, filed on Apr. 27, 2005.

(30) Foreign Application Priority Data
Apr. 30, 2004 (JP) 2004-136290

(51) Int. Cl. A61B 18/18

(52) U.S. Cl. 606/3; 340/573.4; 340/5.8

(57) ABSTRACT

In an energy irradiation device, individual identification information provided to an energy transmitting and irradiating section is acquired, and a date and time when the individual identification information has been acquired is recorded. Next, the acquired individual identification information is associated with at least one of an output signal of an output operating section and a date and time when the information has been recorded to store the information. When new individual identification information is acquired, the new individual identification information is searched from the individual identification information stored in the memory. It is judged whether or not energy irradiation is possible based on the information of the output signal or the date and time stored in association with the individual identification information in a case where the new individual identification information exists in the individual identification information stored in the memory as a result of the searching.

### CROSS REFERENCE TO RELATED APPLICATIONS

This is a Continuation Application of PCT Application No. PCT/JP2005/008046, filed Apr. 27, 2005, which was published under PCT Article 21(2) in Japanese.

This application is based upon and claims the benefit of priority from prior Japanese Patent Application No. 2004-136290, filed Apr. 30, 2004, the entire contents of which are incorporated herein by reference.

### BACKGROUND OF THE INVENTION

1. Field of the Invention

The present invention relates to an energy irradiation device and an energy irradiation method in which, for example, energy is irradiated to a living tissue or the like.

2. Description of the Related Art

An energy irradiation device includes an energy transmitting and irradiating section called an applicator for transmitting energy of laser light or the like generated by an energy generation unit. As to this energy transmitting and irradiating section, the following improvement technology is proposed in Jpn. Pat. Appln. KOKAI Publication No. 2003-10232. That is, attention is focused on a point that a transmission efficiency of energy fluctuates with individual energy transmitting and irradiating section, and individual identification numbers of the energy transmitting and irradiating sections are input, and the used individual identification number is accumulated and recorded. When a new individual identification number is input, the number is compared with recorded use history, and the energy transmitting and irradiating section is prohibited from being used in a case where the same individual identification number exists. This can prevent reuse of the energy transmitting and irradiating section.

### BRIEF SUMMARY OF THE INVENTION

According to a first aspect of the present invention, there is provided an energy irradiation device comprising:

an energy generating section which generates energy;

an output operating section which generates a control signal to instruct the energy generating section to generate the energy;

an energy transmitting and irradiating section which is detachably attached to the energy generating section and which transmits and irradiates the energy;

an acquiring section which acquires individual identification information provided to the energy transmitting and irradiating section;

a date and time recording section which records a date and time when the acquiring section acquires the individual identification information;

a memory which associates the individual identification information acquired by the acquiring section with at least one of an output signal of the output operating section and the date and time recorded by the date and time recording section to store the information;

a searching section which searches for new individual identification information from the individual identification information stored in the memory at a time when the acquiring section acquires the new individual identification information; and

a control section which judges whether or not energy irradiation is possible based on the information of the output signal or the date and time stored in association with the individual identification information in a case where the new individual identification information exists in the individual identification information stored in the memory as a result of the searching by the searching section.

According to a second aspect of the present invention, there is provided an energy irradiation device according to the first aspect, wherein the memory associates, with the individual identification information, the number of energy irradiations by the energy transmitting and irradiating section to store the information, and the control section judges whether or not the energy irradiation is possible based on the information of the control signal or the date and time and the number of the energy irradiations in a case where the new individual identification information exists in the individual identification information stored in the memory as a result of the searching by the searching section.

According to a third aspect of the present invention, there is provided an energy irradiation device according to the first or second aspect, wherein the individual identification information includes information of an energy transmission efficiency of the energy transmitting and irradiating section,

the device further comprising:

a setting section which sets an irradiation energy value irradiated from the energy transmitting and irradiating section;

a calculating section which calculates the energy generation value generated by the energy generating section based on the individual identification information and the irradiation energy value; and

an adjusting section which adjusts an energy generation amount of the energy generating section based on the calculated energy generation value.

According to a fourth aspect of the present invention, there is provided an energy irradiation device according to the first aspect, wherein it is judged that the energy irradiation is possible regardless of the result of the searching by the searching section in a case where predetermined individual identification information is acquired.

According to a fifth aspect of the present invention, there is provided an energy irradiation method comprising the steps of:

acquiring individual identification information provided to an energy transmitting and irradiating section to identify the energy transmitting and irradiating section which transmits and irradiates generated energy;

recording a date and time when the individual identification information is acquired;

associating, with the acquired individual identification information, at least one piece of information on a date and time when the individual identification information has been recorded, whether or not energy has been output, and the number of energy uses to store the information;

searching for new individual identification information from the individual identification information stored in the memory at a time when the new individual identification information has been acquired; and

judging whether or not energy irradiation is possible based on the information stored in association with the individual identification information in a case where the new individual identification information exists in the stored individual identification information as a result of the searching.

According to a sixth aspect of the present invention, there is provided an energy irradiation method according to the fifth aspect, wherein the information stored in association with the individual identification information includes information on whether or not the previous energy use has been discontinued.

According to a seventh aspect of the present invention, there is provided an energy irradiation method according to the fifth or sixth aspect, further comprising the step of:

judging whether or not the new individual identification information is predetermined individual identification information prior to searching of the acquired new individual identification information from the individual identification information stored in the memory.

### BRIEF DESCRIPTION OF THE SEVERAL VIEWS OF THE DRAWING

FIG. 1 is a constitution diagram of a heating system to which a first embodiment of the present invention is applied;

FIG. 2 is an enlarged diagram showing an inserting portion 2 of an applicator 1;

FIG. 3 is a block diagram showing a constitution of the heating system shown in FIG. 1;

FIG. 4 is a diagram showing a recording example of a use individual identification number recorded in a memory 21;

FIG. 5 is a diagram showing a display screen of an operation and display unit 10 at a time when an individual identification number is input in a preparatory stage;

FIG. 6 is a diagram showing a display screen of an operation and display unit 10 at a time when treatment conditions are set;

FIG. 7 is a flowchart (No. 1) showing a function of the first embodiment of the present invention;

FIG. 8 is a flowchart (No. 2) showing a function of the first embodiment of the present invention;

FIG. 9 is a flowchart showing a function of a second embodiment of the present invention;

FIG. 10 is a flowchart showing a function of a third embodiment of the present invention; and

FIG. 11 is a flowchart showing a function of a fourth embodiment of the present invention.

### DETAILED DESCRIPTION OF THE INVENTION

Embodiments of the present invention will be described hereinafter in detail with reference to the drawings.

### First Embodiment

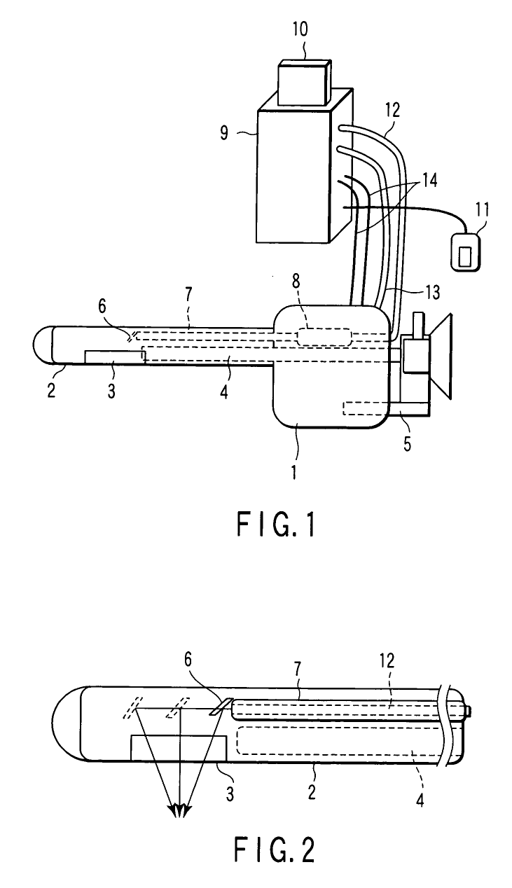

FIG. 1 is a constitution diagram of a heating system to which a first embodiment of the present invention is applied. A main body 9 can be connected to an applicator 1 as an energy transmitting and irradiating section via an optical fiber 12, a signal cable 13, and a cooling water tube 14.

The applicator 1 includes an inserting portion 2 to be inserted into, for example, patient's urethra for a heating treatment of enlarged prostate. An optical fiber protecting tube 7 is disposed in the inserting portion 2, and a laser reflective mirror 6 is connected to a distal end of the tube. The optical fiber 12 is fixed in the optical fiber protecting tube 7.

A laser beam generated in the main body 9 and transmitted via the optical fiber 12 hits the laser reflective mirror 6, and is reflected downwards. The reflected laser beam is irradiated to a living tissue (not shown) brought into contact with a laser irradiation window 3.

The optical fiber protecting tube 7 is movable forwards and backwards by a driving section 8 controlled by the main body 9 via the signal cable 13. The laser reflective mirror 6 irradiates the laser beam while moving in conjunction with the movement of the optical fiber protecting tube 7. Cooling water from the main body 9 is circulated through the inserting portion 2 via the cooling water tube 14, and this prevents heat injury of the living tissue which comes into contact with the laser irradiation window 3.

A temperature sensor (not shown) is disposed in the vicinity of the laser irradiation window 3 in order to measure a temperature of the living tissue of a portion to which the laser beam is irradiated. An endoscope 4 is inserted into the applicator 1, and held with a fixing piece 5 so that the living tissue can be observed from the laser irradiation window 3.

An operation and display unit (monitor provided with a touch panel) 10 is disposed in the main body 9. Furthermore, the main body 9 can be connected to a foot switch 11 as an output operating section which generates a control signal for controlling ON/OFF of an output of laser.

FIG. 2 is an enlarged diagram showing the inserting portion 2 of the applicator 1. It is shown that the laser reflective mirror 6 reciprocates while changing a reflection angle in conjunction with the movement of the optical fiber protecting tube 7. The optical fiber protecting tube 7 is reciprocated to thereby condense the laser beam irradiated from the laser irradiation window 3.

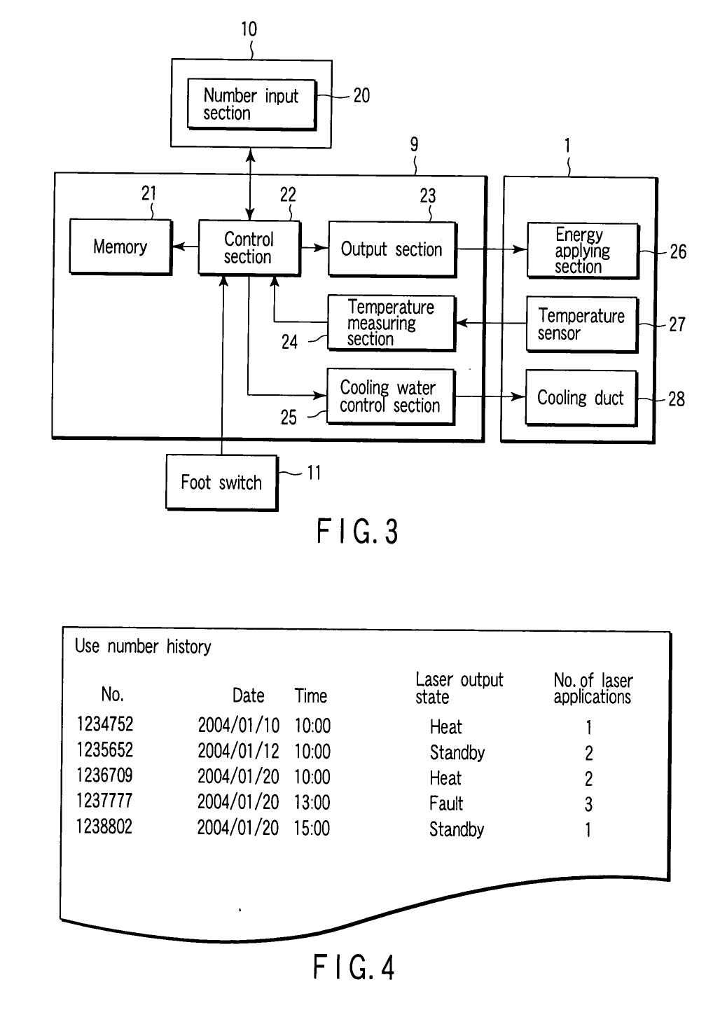

FIG. 3 is a block diagram showing a constitution of the heating system shown in FIG. 1. The main body 9 has a control section 22, a memory 21, an output section 23, a temperature measuring section 24, and a cooling water control section 25. The applicator 1 has an energy irradiating section 26, a temperature sensor 27, and a cooling duct 28. Furthermore, the operation and display unit 10 includes a number input section 20 for inputting an applicator individual identification number (hereinafter referred to as the individual identification number) as individual identification information.

The individual identification number input via the number input section 20 is transmitted to the control section 22. The control section 22 accumulates the input individual identification number and relevant information in the memory 21. The laser beam generated by the output section 23 is provided to the energy irradiating section 26 of the applicator 1.

On the other hand, temperature data detected by the temperature sensor 27 of the applicator 1 is sent to the temperature measuring section 24, and measured, and a measured value is sent to the control section 22. Cooling water from the cooling water control section 25 is sent to the cooling duct 28 of the applicator 1.

Moreover, the control section 22 controls an ON/OFF state of a laser output of the output section 23 based on a control signal generated by operating the foot switch 11.

FIG. 4 is a diagram showing a recording example (use number history) of a use individual identification number recorded in the memory 21. Relevant data such as date and time is recorded by a row unit every individual identification number. The individual identification number is constituted of a serial number as individual information of a product, efficiency data indicating a laser transmission efficiency to the energy irradiating section 26 every individual, and a check sum for confirming whether or not there has been an erroneous input. For example, in “1234752” which is an individual identification number of an uppermost row, “1234” is the serial number, “75” is the efficiency data, and “2” is the check sum. In this case, the efficiency data indicates a percentage numerical value, but may be a group number obtained by dividing data into several grades of groups by the efficiency. The check sum can be variously considered. Here, the check sum is a lower digit (“2”) of numeral “22” obtained by adding up numerals of the serial number “1234” and the efficiency data “75”.

As shown in FIG. 4, information to be recorded every individual identification number includes a date and time on which an input is first made, an output state record indicating whether or not a laser output has been performed, the number of laser uses, and the like. Examples of the output state record include Standby indicating a state in which preparation is made, cooling water is simply circulated, and any laser output is not performed; Heat indicating a state in which the laser output has been performed; and Fault indicating a state in which an error is generated, and the operation has been stopped.

It is to be noted that all, one or two of the date and time, the laser output state, and the number of the laser outputs may be recorded in association with the individual identification number.

FIG. 5 is a diagram showing a display screen of the operation and display unit 10 at a time when the individual identification number is input in a preparatory stage. This display screen is displayed, when a “treatment” button of a menu screen (not shown) is pressed after turning on the power supply.

In the preparatory stage, the main body 9 has to be connected to the optical fiber 12, the signal cable 13, the cooling water tube 14 and the like. Here, as a check list of necessary works, there is an item display (check item 1, check item 2, check item 3, . . . ) 33. When an explanation button 34 disposed corresponding to each item is pressed, a method of the operation or the like is displayed as item explanation 31. At this time, a work position 35 is displayed on an illustration 40 of the main body 9.

Furthermore, the number input section 20 for inputting the individual identification number is disposed, and the individual identification number recorded on the applicator itself or a packing box is input using a soft keyboard 36. FIG. 5 shows a case where “1234561” has been input as the individual identification number. The number input by pressing “Enter” is brought into a determined state. Furthermore, there are disposed a temperature display 30 of the temperature sensor 27, and shift switches 32-1, 32-2 for shifting a treatment step. The shift switch 32-1 is a switch for returning to the previous step, and the shift switch 32-2 is a switch for advancing to the next step.

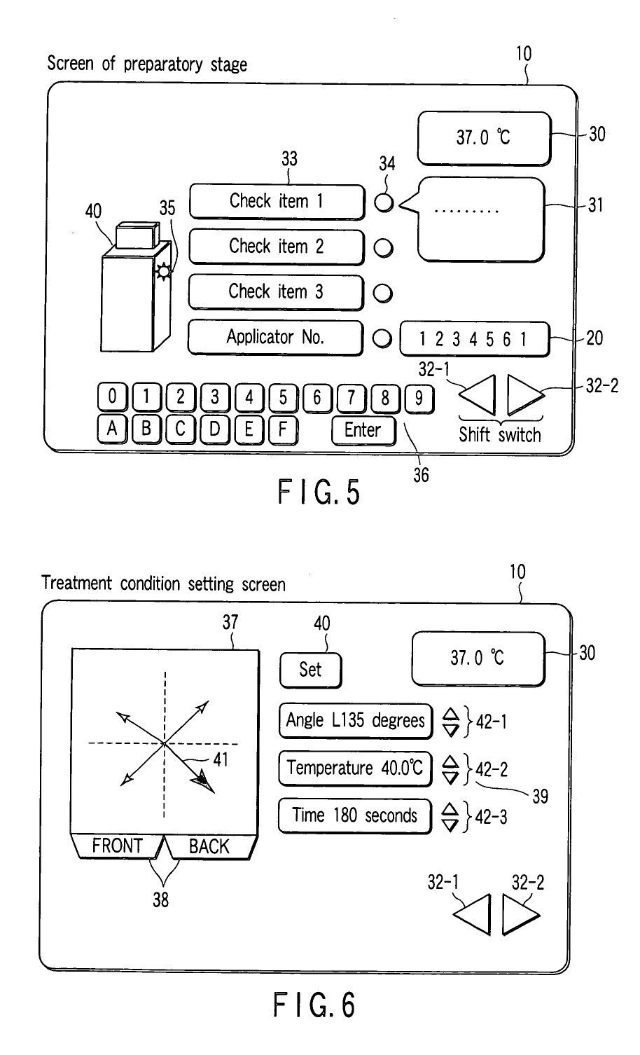

FIG. 6 is a diagram showing a display screen of the operation and display unit 10 at a time when treatment conditions are set. This display screen is displayed, when the shift switch 32-2 is pressed.

When a set button 40 is pressed, an arrow 41 indicating an irradiating direction of laser energy is displayed on a set image 37. A user sets a rotary angle of an energy irradiating direction by use of a change button 42-1 of a setting section 39. A heating temperature is set using a change button 42-2. A heating time is set using a change button 42-3.

The set image 37 has two layers so that an applicator insertion length can be distinguished and set with respect to urethra, and FRONT and BACK can be switched by a changeover button 38. For example, for the treatment of the more enlarged prostate, the urethra insertion length is divided into two stages and the laser is then irradiated to the enlarged prostate to improve a treatment effect for a patient.

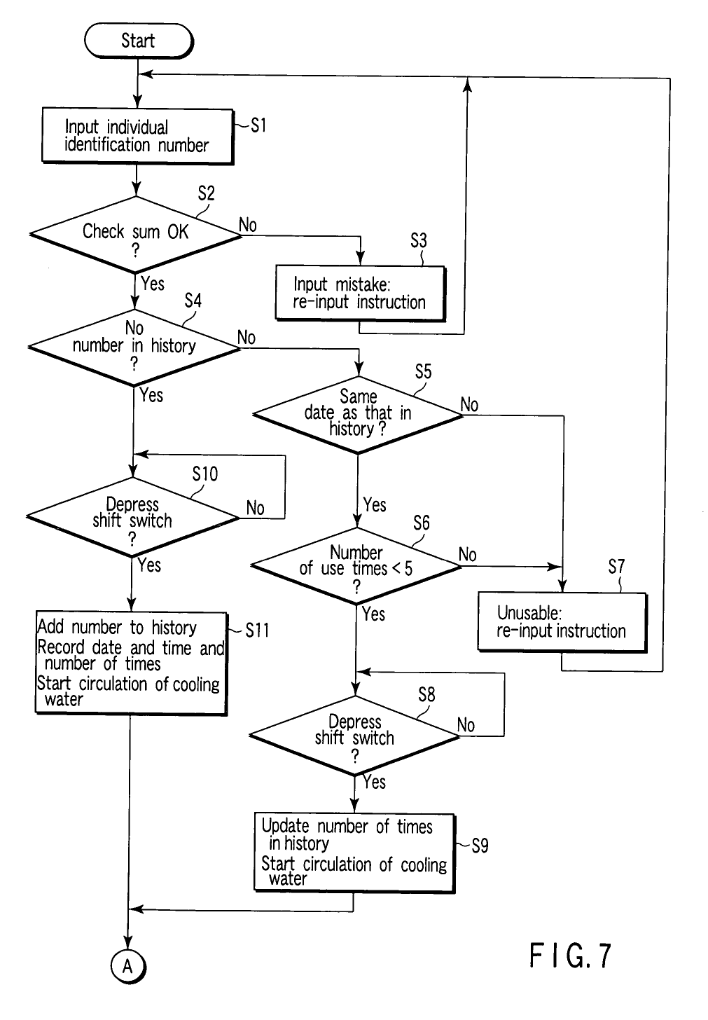

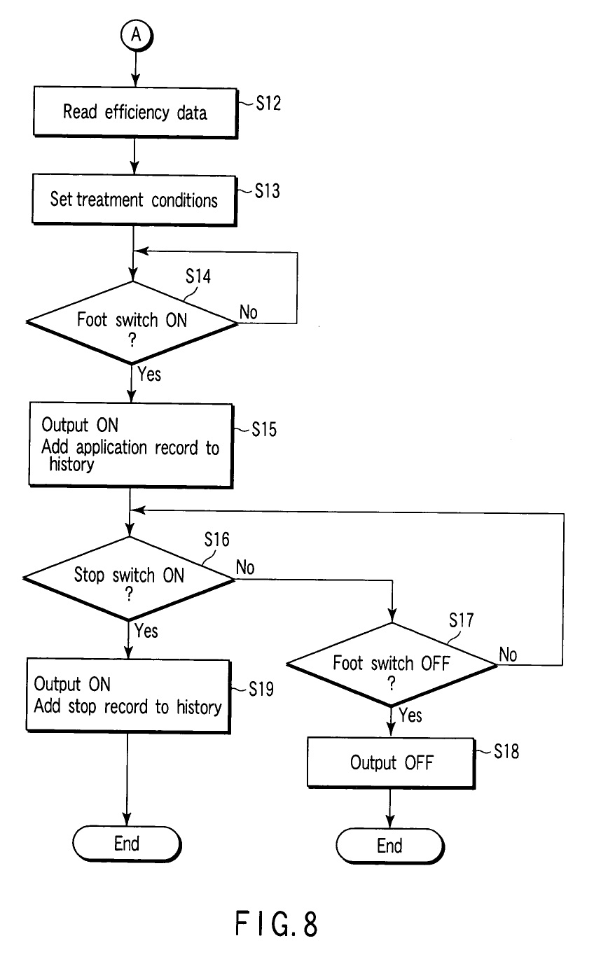

FIGS. 7 and 8 are flowcharts showing a function of the first embodiment. When the individual identification number is input (step S1), a check sum portion of the number is checked to thereby judge whether or not there is an input mistake (step S2). When check sum does not agree (NO in the judgment of the step S2), it is judged that the input is a mistake, and the user is instructed to re-input the number by dialog display or emission of sound (step S3). Thereafter, the flow returns to the step S1.

When the check sum agrees (YES in the judgment of the step S2), it is checked whether or not the input individual identification number exists in a use number history (FIG. 4) (step S4).

When the individual identification number does not exist (YES in the judgment of the step S4), it is waited until the shift switch 32-2 for advancing to the next processing is depressed (step S10). When the shift switch 32-2 is depressed (YES in the judgment of the step S10), the present number, the date and time, the output state=Standby, and the number of uses=1 are additionally recorded in the use number history, and the cooling water control section 25 starts circulation of cooling water through the applicator 1 (step S11). That is, it is seen whether or not cooling water has been circulated by judging whether or not the record exists in the use number history.

On the other hand, in a case where the input individual identification number exists in the use number history (NO in the judgment of the step S4), it is judged whether or not the date recorded in the history is the same as the present date (step S5). When the date is different (NO in the judgment of the step S5), it is judged that the number cannot be reused, and the user is instructed to re-input the number by the dialog display or the emission of sound (step S7). Thereafter, the flow returns to the step S1.

On the other hand, in a case where the date recorded in the history is the same as the present date (YES in the judgment of the step S5), it is confirmed whether or not the number of the uses recorded in the history is below five (step S6). In a case where the number is already used five times (NO in the judgment of the step S6), it is judged that the number cannot be reused, and the user is instructed to re-input the number by the dialog display or the emission of sound (step S7).

Moreover, when the number of the uses is less than five (YES in the judgment of the step S6), it is waited until the shift switch 32-2 of a treatment step is depressed (step S8). When the shift switch 32-2 is depressed (YES in the judgment of the step S8), the number of the uses in the use number history is updated, and the cooling water control section 25 starts the circulation of cooling water through the applicator 1 (step S9).

Next, efficiency date is read from the input individual identification number (step S12), and setting of laser output of the output section 23 is adjusted in accordance with the read efficiency date. That is, an energy generation value is calculated based on the read efficiency date and an irradiation energy value set via the operation and display unit 10. When the output is turned on in step S15 described later, an amount of energy to be generated by the output section 23 is adjusted to obtain this energy generation value. Details of this processing are described in paragraphs “0050” to “0055” of Jpn. Pat. Appln. KOKAI Publication No. 2003-10232 described above.

When the laser irradiation has been prepared (step S13), it is judged whether or not the foot switch 11 has been turned on (step S14). When the foot switch 11 is turned on (YES in the judgment of the step S14), the laser output is started, and the output state in the use number history is updated to “Heat” to record the laser irradiation (step S15).

Moreover, it is judged whether or not a stop switch (not shown) has been depressed for a reason such as an error (step S16). When the switch is depressed (YES in the judgment of the step S16), the laser output is turned off, and the output state in the use number history is updated to “Fault” to record the stop (step S19), thereby ending the flow. When the stop switch is not depressed (NO in the judgment of the step S16), it is judged whether or not the foot switch 11 has been turned off (step S17). Here, when the foot switch 11 is turned off (YES in the judgment of the step S17), the laser output is turned off (step S18), thereby ending the flow. When the foot switch 11 is not turned off (NO in the judgment of the step S17), the flow returns to the step S16).

According to the above-described first embodiment, the following effect is produced. In a case where the setting is performed again from the beginning for a certain reason, it is supposed that the setting is performed on the same date. Therefore, record date of the individual identification number can be compared with input date to thereby reuse the number in a case where the power supply is once turned off for a certain reason. An upper limit of the number of the uses can be disposed to avoid abnormal use.

### Second Embodiment

A second embodiment of the present invention will be described hereinafter. Since a constitution of the second embodiment is similar to that of the first embodiment, description thereof is omitted here.

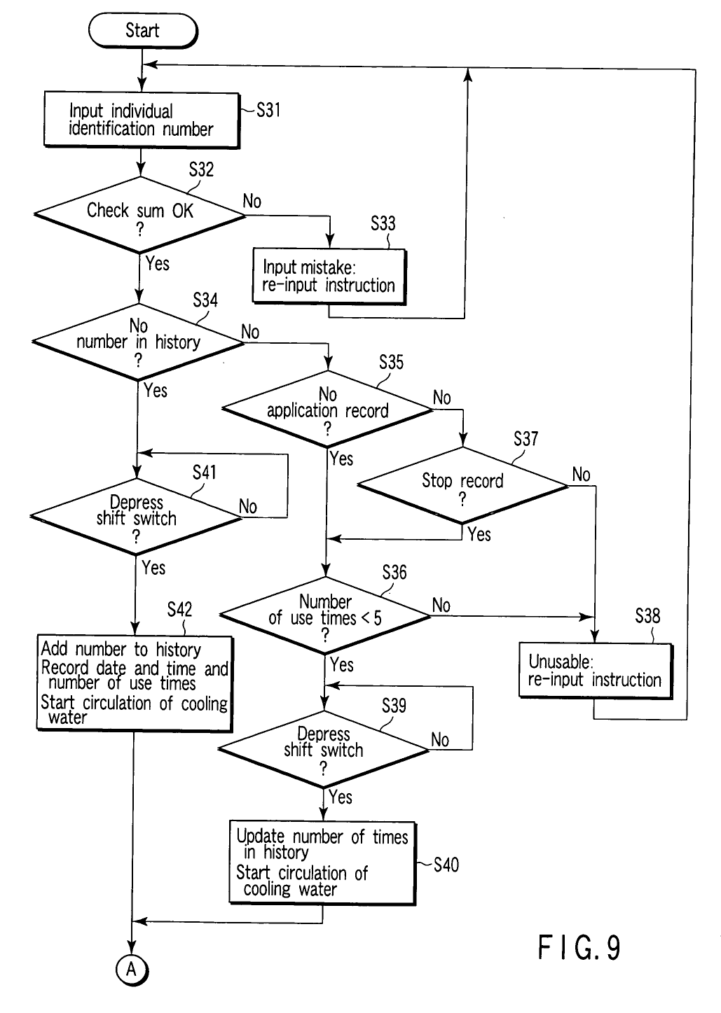

FIG. 9 is a flowchart showing a function of the second embodiment. When an individual identification number is input (step S31), a check sum is confirmed to judge whether or not there is an input mistake (step S32). When the check sum does not agree (NO in the judgment of the step S32), it is judged that the input is a mistake, and a user is instructed to re-input the number by dialog display or emission of sound (step S33). Thereafter, the flow returns to the step S31.

When the check sum agrees (YES in the judgment of the step S32), it is checked whether or not the individual identification number exists in a use number history (step S34). When the individual identification number does not exist (YES in the judgment of the step S34), it is waited until a treatment step shift switch 32-2 is depressed (step S41). When the shift switch 32-2 is depressed (YES in the judgment of the step S41), the present number, a date and time, an output state=Standby, and the number of uses=1 are additionally recorded in the use number history (step S42).

On the other hand, in a case where the input individual identification number exists in the use number history (NO in the judgment of the step S34), it is judged whether or not the laser irradiation record exists (step S35). When the output state in the history is “Standby”, it is judged that the laser irradiation record does not exist (YES in the judgment of the step S35), and the flow advances to step S36. In a case where the output state in the history is other than “Standby”, it is judged that there exists the laser irradiation record (NO in the judgment of the step S35), and it is confirmed whether or not the state is stop record “Fault” (step S37).

In case of “Fault”, it is judged that there is the stop record (YES in the judgment of the step S37), and the flow advances to step S36. In a case where the state is not “Fault”, it is judged that the previous treatment has been normally completed (NO in the judgment of the step S37), it is judged that the number cannot be reused, and a user is instructed to re-input the number by dialog display or emission of sound (step S38). Thereafter, the flow returns to the step S31.

In step S36, the number of the uses recorded in the history is confirmed. In a case where the number is already used five times (NO in the judgment of the step S36), it is judged that the number cannot be reused, and the user is instructed to re-input the number by the dialog display or the emission of sound (step S38). When the number of uses is less than five (YES in the judgment of the step S36), it is waited until the treatment step shift switch 32-2 is depressed (step S39). When the shift switch 32-2 is depressed (YES in the judgment of the step S39), the number of the uses in the use number history is updated, and circulation of cooling water is started (step S40). The subsequent flow is the same as that of the first embodiment as shown in FIG. 8.

According to the above second embodiment, when it is judged whether or not the treatment has been normally completed by judging whether or not the previous use is completed with a stop button, reuse in a case where set-up is performed again owing to a certain error is possible. When an upper limit of the number of the uses is disposed, abnormal use can be avoided.

### Third Embodiment

A third embodiment of the present invention will be described hereinafter. Since a constitution of the third embodiment is similar to that of the first embodiment, description thereof is omitted here.

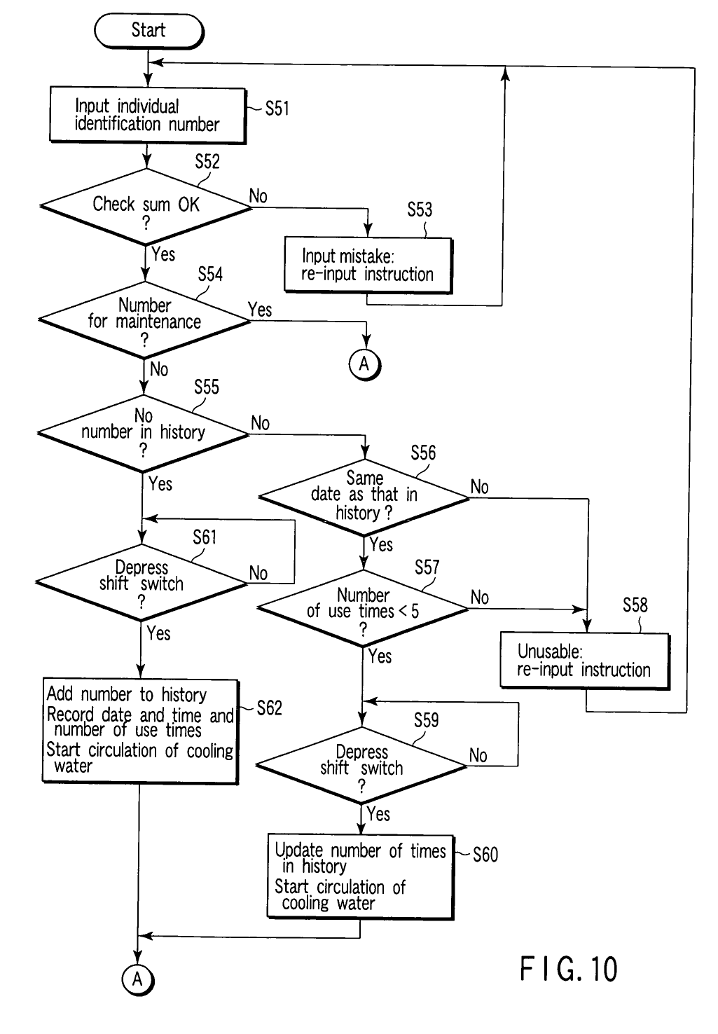

FIG. 10 is a flowchart showing a function of the third embodiment. When an individual identification number is input (step S51), a check sum is confirmed to judge whether or not there is an input mistake (step S52). When the check sum does not agree (NO in the judgment of the step S52), it is judged that the input is a mistake, and a user is instructed to re-input the number by dialog display or emission of sound (step S53). Thereafter, the flow returns to the step S51.

When the check sum agrees (YES in the judgment of the step S52), it is judged whether or not the number is a predetermined number for maintenance (step S54). In a case of the predetermined number for maintenance (YES in the judgment of the step S54), the number becomes usable regardless of presence of history, and the flow advances to FIG. 8. The subsequent flow is the same as that of the first embodiment. It is to be noted that as an example of a maintenance work, there is confirmation of device output precision, temperature measurement precision or the like.

On the other hand, in a case where the number is not for maintenance (NO in the judgment of the step S54), it is judged whether or not the individual identification number exists in a use number history (step S55). When the individual identification number does not exist (YES in the judgment of the step S55), it is waited until a treatment step shift switch 32-2 is depressed (step S61). When the shift switch 32-2 is depressed (YES in the judgment of the step S61), the present number, date and time, output state=Standby, and the number of uses=1 are additionally recorded in the use number history, and a cooling water control section 25 starts circulation of cooling water through an applicator 1 (step S62). That is, it is seen whether or not cooling water has been circulated by judging whether or not a record exists in the use number history.

On the other hand, in a case where the input individual identification number exists in the use number history (NO in the judgment of the step S55), it is judged whether or not the date recorded in the history is the same as the present date (step S56). In a case where the date differs (NO in the judgment of the step S56), it is judged that the number cannot be reused, and the user is instructed to re-input the number by the dialog display or the emission of sound (step S58). In a case where the date is the same day (YES in the judgment of the step S56), it is confirmed whether or not the number of the uses recorded in the history is less than five (step S57). In a case where the number is already used five times (NO in the judgment of the step S57), it is judged that the number cannot be reused, and the user is instructed to re-input the number by the dialog display or the emission of sound (step S58). Thereafter, the flow returns to the step S51.

Moreover, when the number of the uses is less than five (YES in the judgment of the step S57), it is waited until the treatment step shift switch 32-2 is depressed (step S59). When the shift switch 32-2 is depressed (YES in the judgment of the step S59), the number of the uses in the use number history is updated, and circulation of cooling water is started (step S60).

According to the above third embodiment, in addition to the effects of the first and second embodiments, a work during maintenance can be easily performed.

### Fourth Embodiment

A fourth embodiment of the present invention will be described hereinafter. Since a constitution of the fourth embodiment is similar to that of the first embodiment, description thereof is omitted here.

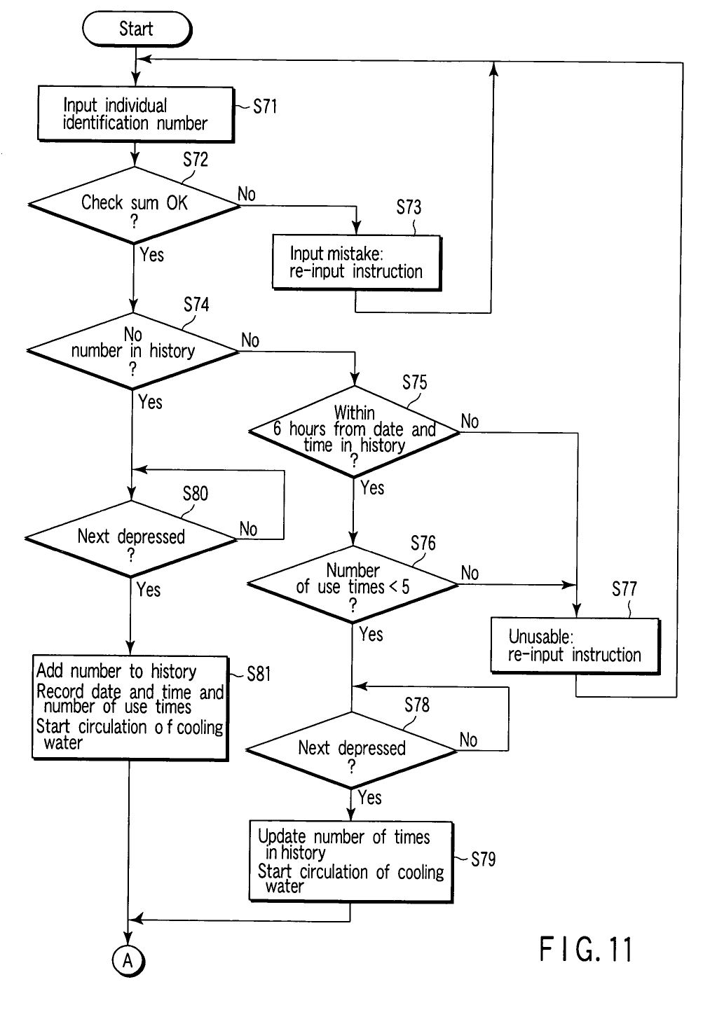

FIG. 11 is a flowchart showing the fourth embodiment. When an individual identification number is input (step S71), a check sum portion of the number is checked to judge whether or not there is an input mistake (step S72). When the check sum does not agree (NO in the judgment of the step S72), it is judged that the input is a mistake, and a user is instructed to re-input the number by dialog display or emission of sound (step S73). Thereafter, the flow returns to the step S71.

When the check sum agrees (YES in the judgment of the step S72), it is checked whether or not the input individual identification number exists in a use number history (step S74).

When the individual identification number does not exist (YES in the judgment of the step S74), it is waited until a shift switch 32-2 for advancing to the next processing is depressed (step S80). When the shift switch 32-2 is depressed (YES in the judgment of the step S80), the present number, date and time, output state=Standby, and the number of uses=1 are additionally recorded in the use number history, and a cooling water control section 25 starts circulation of cooling water through an applicator 1 (step S81). That is, it is seen whether or not cooling water has been circulated by judging whether or not the record exists in the use number history.

On the other hand, in a case where the input individual identification number exists in the use number history (NO in the judgment of the step S74), it is judged whether or not an elapsed time from a date and time recorded in the history up to now is within six hours (step S75). When the elapsed time up to now is above six hours (NO in the judgment of the step S75), it is judged that the number cannot be reused, and the user is instructed to re-input the number by the dialog display or the emission of sound (step S77). Thereafter, the flow returns to the step S71.

On the other hand, in a case where the elapsed time from the date and time recorded in the history up to now is within six hours (YES in the judgment of the step S75), it is confirmed whether or not the number of the uses recorded in the history is less than five (step S76). In a case where the number is already used five times (NO in the judgment of the step S76), it is judged that the number cannot be reused, and the user is instructed to re-input the number by the dialog display or the emission of sound (step S77).

When the number of the uses is less than five (YES in the judgment of the step S76), it is waited until the shift switch 32-2 for advancing to the next processing is depressed (step S78). When the shift switch 32-2 is depressed (YES in the judgment of the step S78), the number of the uses in the use number history is updated, and the cooling water control section 25 starts the circulation of cooling water through the applicator 1 (step S79). The subsequent flow is similar to that of the first embodiment as shown in FIG. 8.

It is to be noted that as an example of the step S75, it is judged that the elapsed time is within six hours, but this time may be shorter or longer.

According to the above fourth embodiment, if the date changes in a case where the treatment is performed in the night in the first embodiment, the targeted effect cannot be obtained. When it is judged whether or not the reuse is possible by the time from the record date of the individual identification number as in the present embodiment, reuse in a case where a power supply is once turned off is possible in any time zone. Conditions of the number of the uses can be provided to avoid abnormal use.

(Note)

The inventions constituted as follows can be extracted from the above-described typical embodiments.

1. An energy irradiation device comprising:

an energy generating section which generates energy;

an output operating section which instructs the energy generating section to generate the energy;

an energy transmitting and irradiating section which is detachably attached to the energy generating section and which transmits and irradiates the energy;

an acquiring section which acquires individual identification information provided to the energy transmitting and irradiating section;

a date and time recording section which records a date and time when the acquiring section acquires the individual identification information;

a storage section which associates the individual identification information acquired by the acquiring section with at least one of an output signal of the output operating section and the date and time recorded by the date and time recording section to store the information;

a searching section which searches for new individual identification information from the individual identification information stored in the storage unit at a time when the acquiring section acquires the new individual identification information; and

a control section which judges whether or not energy irradiation is possible based on the information of the output signal or the date and time stored in association with the individual identification information in a case where the new individual identification information exists in the individual identification information stored in the storage unit as a result of the searching by the searching section.

2. The energy irradiation device according to 1, wherein the individual identification information includes a serial number as individual information of a product, efficiency data indicating a laser transmission efficiency of each individual including up to the energy transmitting and irradiating section, and a check sum to confirm whether or not an erroneous input has been performed.

3. The energy irradiation device according to 1, wherein the storage section further stores information on an energy output state including a state in which any energy is not output, a state in which the energy is output, and a state in which the energy output is stopped.

4. The energy irradiation device according to 1, further comprising:

a section which records the individual identification information and which supplies a fluid for cooling,

wherein it is judged whether or not the fluid for cooling has been provided by judging whether or not there is any record of the individual identification information.

According to the present invention, it is possible to reuse an energy transmitting and irradiating section if necessary, and the reuse of the energy transmitting and irradiating section can be managed in a mode suitable for circumstances in a medical field. That is, an operator can perform treatment irrespective of a use method (usability) of the energy transmitting and irradiating section designated by a maker.

1. An energy irradiation device comprising:
an energy generating section which generates energy;
an output operating section which generates a control signal to instruct the energy generating section to generate the energy;
an energy transmitting and irradiating section which is detachably attached to the energy generating section and which transmits and irradiates the energy;
an acquiring section which acquires individual identification information provided to the energy transmitting and irradiating section;
a date and time recording section which records a date and time when the acquiring section acquires the individual identification information;
a memory which associates the individual identification information acquired by the acquiring section with at least one of an output signal of the output operating section and the date and time recorded by the date and time recording section to store the information;
a searching section which searches for new individual identification information from the individual identification information stored in the memory at a time when the acquiring section acquires the new individual identification information; and
a control section which judges whether or not energy irradiation is possible based on the information of the output signal or the date and time stored in association with the individual identification information in a case where the new individual identification information exists in the individual identification information stored in the memory as a result of the searching by the searching section.

2. The energy irradiation device according to claim 1, wherein the memory associates, with the individual identification information, the number of energy irradiations by the energy transmitting and irradiating section to store the information, and the control section judges whether or not the energy irradiation is possible based on the information of the control signal or the date and time and the number of the energy irradiation in a case where the new individual identification information exists in the individual identification information stored in the memory as a result of the searching by the searching section.

3. The energy irradiation device according to claim 1, wherein the individual identification information includes information of an energy transmission efficiency of the energy transmitting and irradiating section,
the device further comprising:
a setting section which sets an irradiation energy value irradiated from the energy transmitting and irradiating section:
a calculating section which calculates the energy generation value generated by the energy generating section based on the individual identification information and the irradiation energy value; and
an adjusting section which adjusts an energy generation amount of the energy generating section based on the calculated energy generation value.

4. The energy irradiation device according to claim 1, wherein it is judged that the energy irradiation is possible regardless of the result of the searching by the searching section in a case where predetermined individual identification information is acquired.

5. An energy irradiation method comprising the steps of:
acquiring individual identification information provided to an energy transmitting and irradiating section to identify the energy transmitting and irradiating section which transmits and irradiates generated energy:
recording a date and time when the individual identification information is acquired;
associating, with the acquired individual identification information, at least one piece of information on a date and time when the individual identification information has been recorded, whether or not energy has been output, and the number of energy uses to store the information;
searching for new individual identification information from the individual identification information stored in the memory at a time when the new individual identification information has been acquired; and
judging whether or not energy irradiation is possible based on the information stored in association with the individual identification information in a case where the new individual identification information exists in the stored individual identification information as a result of the searching.

6. The energy irradiation method according to claim 5, wherein the information stored in association with the individual identification information includes information on whether or not the previous energy use has been discontinued.

7. The energy irradiation method according to claim 5, further comprising the step of:
judging whether or not the new individual identification information is predetermined individual identification information prior to searching of the acquired new individual identification information from the individual identification information stored in the memory.
